(19) United States
(12) Patent Application Publication
(10) Pub. No.: US 2022/0323795 A1
(43) Pub. Date: Oct. 13, 2022

(54) COMBINATION OF CICLOPIROXOLAMINE AND PIROCTONE OLAMINE FOR COMBATING DANDRUFF

(71) Applicant: PIERRE FABRE DERMO-COSMETIQUE, Boulogne-Billancourt (FR)

(72) Inventors: Joëlle LUC, FENOUILLET (FR); Carole MICHEL, MOURVILLES-BASSES (FR)

(73) Assignee: PIERRE FABRE DERMO-COSMETIQUE, Boulogne-Billancourt (FR)

(21) Appl. No.: 17/764,836

(22) PCT Filed: Sep. 29, 2020
(86) PCT No.: PCT/FR2020/051698

§ 371 Date: Mar. 29, 2022

(30) Foreign Application Priority Data
Sep. 30, 2019 (FR) FR1910808

(51) Int. Cl. A61Q 5/00; A61K 8/49

(52) U.S. Cl. CPC A61Q 5/006; A61K 8/4926

(57) ABSTRACT

The present invention relates to the use of a combination of ciclopiroxolamine and piroctone olamine without zinc pyrithione or of a dermatological or cosmetic composition comprising such a combination for combating dandruff and/or preventing the formation thereof.

### FIELD OF THE INVENTION

The present invention relates to a combination of ciclopiroxolamine and of piroctone olamine and the compositions containing this combination for their application in the field of cosmetics and dermatology for fighting against dandruff.

### PRIOR ART

Dandruff affects one in two people in France. Its presence is benign, but it gives an unesthetic appearance.

Dandruff consists of flakes of skin and, more precisely, surface cells of the scalp that have flaked off, but which are unusually large in size, thicker or more numerous than normal cells. The scalp, like all skin, normally renews itself by removing surface cells from the epidermis. Dandruff is said to be dry or oily.

Dry dandruff is visible to the naked eye and appears as fragments of the stratum corneum of the scalp. It is white, flat and detaches spontaneously. It is often accompanied by unpleasant itching. It is generally paired with a dry scalp.

Oily dandruff is instead small, round, yellowish and oily It adheres to the scalp or to the hair. There it forms a yellowish and sticky layer. It is accompanied by itchiness. In general, it is the consequence of seborrhoea and therefore often associated with an oily scalp and hair.

Dandruff is a problem with the formation of the epidermis. A healthy scalp presents dead cells every day without them being noticed. This cellular renewal takes place according to a regular cycle of approximately 28 days. During this process, the cells of the epidermis have time to correctly finalise their maturation, to gradually dissociate from one another, and once they have arrived at the surface, to invisibly detach one by one. In the case of dandruff, the division of the cells of the basal layer of the epidermis is too rapid due to inflammation of the scalp. Cellular renewal then takes place in 5 to 14 days. The cells which rise to the surface of the skin do not have time to lose their water and to harden. They reach the surface without being sufficiently dried out, which forms clusters of cells on the scalp, which become visible: this is dandruff.

There are two dandruff states depending on the severity:
Mild and discreet dandruff: this is the small white flakes corresponding to clusters of very volatile dead cells which fall from the scalp and sprinkle the clothing;
Seborrhoeic dermatitis: this involves dandruff combined with a strong inflammation of the scalp causing redness and itching.

Seborrhoeic dermatitis is in fact a common inflammatory skin dermatitis (observed in approximately 3% of the population) presenting in the form of red patches covered with oily yellowish flakes, with varying degrees of itchiness, predominantly in areas rich in sebaceous glands, the sebaceous areas.

The epidermis of the scalp is covered with a lipid film called sebum, the role of which is, inter alia, to protect it against drying out. Malassezia, sometimes also called Pityrosporum ovale, is the name of a yeast belonging to the group of Fungi imperfecti and part of the commensal flora of human beings. Malassezia (M.) have long been known in human pathology. Several species involved in these pathologies are distinguished: M. furfur, M. sympodialis, M. globosa, M. restricta, M. obtusa. Malassezia are lipophilic and keratinophilic yeasts, they are lipid-dependent. It has been established that this fungus is a mildly pathogenic agent for humans M. furfur is present in practically 100% of the population. However, it has also been established that M. furfur is responsible, in approximately 3% of the population, for dermatoses such as adult seborrhoeic dermatitis. In healthy subjects, the most commonly encountered species are M. sympodialis, M. globosa and M. restricta, whatever the culture media used, the method of sample collection or the country of the study, M. restricta appears to predominate on the scalp, while M. sympodialis predominates on the trunk and M. globosa is widely spread and evenly distributed over the sebaceous areas. Malassezia grows easily in oily environments, such as the scalp where sebum is secreted in particularly high quantities, thus creating a favourable environment for its growth. This fungus can “digest” lipids present in sebum and produce fatty acids that are particularly irritating for the skin and scalp. In seborrhoeic dermatitis, the exact pathogenic mechanism remains unclear, however several factors make it possible to state that Malassezia plays a predominant role. Indeed, it has been shown that: a) the proportion of Malassezia is larger in individuals with seborrhoeic dermatitis, as well as on the affected skin when compared with healthy skin in patients suffering from seborrhoeic dermatitis (Tajima and al., J Invest Dermatol, 2008, 128, 345-351) and b) antifungal treatments effectively reduce the symptomatology (Piérard-Franchimont and al., Dermatology, 2001, 202, 171-176). The current hypothesis holds that seborrhoeic dermatitis is not caused by an exaggerated growth of Malassezia, but by an abnormal response of the host to these fungi. One hypothesis proposes that these yeast produce a lipase, the action of which would transform the triglycerides of the sebum into free fatty acids, which themselves cause an inflammation. Indeed, a recent study as shown that the species M. globosa possesses a gene, LP1, the expression of which enables production of a lipase (DeAngelis and al., J Invest Dermatol 2007, 127, 2138-2146).

Produced in too large a quantity, fatty acids can cause an inflammation at the origin of dandruff. Two species, M. globosa and M. restricta appear today to be more closely implicated in the dandruff state. For approximately 50% of individuals, sebum and Malassezia coexist in good harmony. For the remaining 50%, an imbalance can occur, several factors coming into play. People do not all secrete the same quantity of sebum. Some people secrete a sufficiently large quantity to be conducive to the excessive growth of Malassezia and thus to the formation of dandruff. Furthermore, the quantity of sebum varies over time, depending in particular on hormonal secretions. Finally, other factors can promote the appearance of dandruff, such as stress, fatigue, pollution, lack of sunlight, certain diseases such as Parkinson's disease, some drugs, such as neuroleptics, etc.

Dandruff often appears in adolescence at the time of puberty when the secretion of sebum begins. Men appear to be more frequently affected than women, undoubtedly due to the effect of androgen hormones.

Anti-dandruff compositions, particularly in the form of shampoos, have been known and commercially available for a number of years. They generally contain active chemicals such as ketoconazole, zinc pyrithione, octopirox, piroctone olamine, salicylic acid, selenium sulfide and azelaic acid. Certain compounds, in particular zinc pyrithione, are suspected of causing environmental problems, in particular ecotoxicity causing toxicity in fish. It is therefore important to find new active ingredients or new combinations of active ingredients which are environmentally friendly, in other words without zinc pyrithione, while being highly effective and quick acting.

Hence, despite the many options currently available, consumers still have need of novel products to combat dandruff and/or to prevent its formation.

### SUMMARY OF THE INVENTION

Surprisingly, the inventors discovered that the combination of ciclopiroxolamine and piroctone olamine exhibited a synergistic activity on Malassezia furfur in the implemented model.

The present invention therefore aims to provide an effective treatment against dandruff and for preventing and/or treating seborrhoeic dermatitis, and this without zinc pyrithione. This treatment is preferably carried out topically, which enables greater efficacy.

An important feature of the invention is that the combination comprising ciclopiroxolamine and piroctone olamine is devoid of zinc pyrithione.

Indeed, according to the present invention, it must be understood that each time the combination of ciclopiroxolamine and piroctone olamine is mentioned, there is no zinc pyrithione.

The object of the present invention is therefore a combination comprising ciclopiroxolamine and piroctone olamine, without zinc pyrithione, for the, preferably topical, use thereof in preventing and/or treating dandruff, in particular mild and discreet dandruff, and/or for the use thereof in preventing and/or treating seborrhoeic dermatitis.

Another object of the present the invention is the, preferably topical, use of the combination comprising ciclopiroxolamine and piroctone olamine, without zinc pyrithione, for preventing and or treating dandruff, in particular mild and discreet dandruff, and/or for preventing and/or treating seborrhoeic dermatitis.

Another object of the present invention is the use of the combination comprising ciclopiroxolamine and piroctone olamine, without zinc pyrithione, for producing a dermatological or cosmetic, preferably topical, composition, intended for preventing and/or treating dandruff, in particular mild and discreet dandruff.

The invention also relates to a combination according to the invention for producing a dermatological composition for treating and/or preventing seborrhoeic dermatitis.

According to another aspect, the invention relates to use of a combination according to the invention for producing a dermocosmetic composition for combating dandruff and/or preventing its formation.

Advantageously, the invention also relates to a combination comprising, or consisting of, ciclopiroxolamine and piroctone olamine, as an anti-dandruff active ingredient within a dermatological or cosmetic, in particular topical, composition, for preventing or treating dandruff.

Equally advantageously, the invention also relates to a combination comprising, or consisting of, ciclopiroxolamine and piroctone olamine as an anti-dandruff active ingredient within a dermatological or cosmetic, in particular topical, composition for preventing or treating seborrhoeic dermatitis.

In particular, the ciclopiroxolamine and piroctone olamine combination is devoid of zinc pyrithione and, advantageously, said combination is the only anti-dandruff active ingredient of the composition.

Another object of the present invention is a method for preventing and/or treating dandruff comprising the administration, in particular by topical application, of an effective quantity of the combination comprising ciclopiroxolamine and piroctone olamine, without zinc pyrithione to a person in need of same.

This involves, in particular, mild and discreet dandruff.

In the method according to the invention, the method does not provide for the use of zinc pyrithione. According to the invention, the ciclopiroxolamine and piroctone olamine combination is advantageously the only anti-dandruff active ingredient used in the method.

The present invention also relates to a cosmetic method for a treating and/or preventing dandruff, comprising the administration, in particular by topical application, of this combination comprising ciclopiroxolamine and piroctone olamine, without zinc pyrithione.

This involves, in particular, mild and discreet dandruff. In particular, the method does not provide for the use of zinc pyrithione.

Advantageously, according to the invention, the ciclopiroxolamine and piroctone olamine combination is the only anti-dandruff active ingredient of the composition and of the method according to the invention.

According to another aspect, the invention relates to a method for treating and/or preventing seborrhoeic dermatitis, comprising the administration, in particular by topical application to the scalp, of a combination according to the invention or of a composition according to the invention.

### DETAILED DESCRIPTION OF THE INVENTION

Ciclopiroxolamine and piroctone olamine are antifungal substances from the hydroxypyridone family. These compounds are often used in cosmetic products in human medicine, for example piroctone olamine is used at a maximum concentration of 1% (rinseable products) or 0.05% to 0.1% (sufficient concentration in leave-in products). These two substances are very effective and even considered as second-generation anti-dandruff agents. Moreover, they have very low toxicity. They are, furthermore, particularly suitable for the production of anti-dandruff shampoos and haircare products, due to their solubility in aqueous or mixed (water-alcohol) surfactants. Hydroxypyridones have antifungal and also antibacterial properties and show excellent diffusion capabilities in keratin.

Piroctone olamine is the common name of this compound. It is found in literature under the term octopirox. It appears in the form of a salt, combined with ethanolamine, which has also earned it the name piroctone ethanolamine salt. Its CAS number is 68890-66-4.

Ciclopiroxolamine, also called ciclopirox olamine, has CAS number 29342-05-0.

The anti-dandruff action of these two substances is attributed to their antibacterial and antifungal actions. The antifungal action of the hydroxypyridone family has been studied based on the model of ciclopiroxolamine. Given that its antifungal activity has been attributed to the hydroxypyridone group, this mode of action can be extrapolated to the entire family. This mode of action is complex and partially understood. The antifungal activity appears to result from multiple metabolic processes within the targeted cell. Hydroxypyridones have a high affinity for trivalent metal cations such as iron and aluminium. However, this enzymatic cofactor is necessary for the mitochondrial cytochromes which are involved in the transport of electrons, in particular for complex I (NADH ubiquinone oxidoreductase). The neutralisation of iron and aluminium inhibits this metabolic reaction and thus the production of cellular energy. Furthermore, ciclopirox inhibits the activity of catalases and intracellular peroxidases responsible for breaking down the toxic peroxides for the cell. Studies have also shown that ciclopirox inhibits the transmembrane transport mechanisms of yeast. The uptake of nutrients into microorganisms is thus affected. In growing cells, the depletion of essential amino acids and nucleotides leads to a reduction in the synthesis of proteins and nucleic acids. Piroctone olamine also has an inhibiting action on DNA replication. This hydroxypyridone has no direct effect on the replication enzymes, but the drop in the level of ATP makes this synthesis impossible. Ciclopiroxolamine also has anti-inflammatory effects by inhibiting 5-lipoxygenase and cyclooxygenase. Given the complexity of the mode of action of hydroxypyridones, it is unlikely that resistance phenomena will appear.

The use of ciclopiroxolamine or piroctone olamine is rather common and one or the other is found in many products, sometimes combined with other antimicrobial agents, in particular zinc pyrithione. Examples include the dermatological anti-dandruff shampoo Dercos® comprising piroctone olamine and salicylic acid, the anti-dandruff Shapiro Hegor® containing piroctone olamine and zinc pyrithione, or again the shampoo treating seborrhoeic dermatitis of the scalp with ciclopirox (Sebiprox®) or again (STIPROX®). On the other hand, to date, no product without zinc pyrithione, combines ciclopiroxolamine with piroctone olamine.

Dermatological or Cosmetic Composition

The present invention also relates to a composition, in particular a dermatological or cosmetic (e.g. dermocosmetic) composition, for use thereof for preventing and/or treating seborrhoeic dermatitis or for combating dandruff and/or for preventing its formation, in particular mild and discreet dandruff, comprising the combination of ciclopiroxolamine and piroctone olamine, as defined above, with a dermatologically or cosmetically acceptable excipient, more particularly acceptable for a topical application.

The composition is thus devoid of zinc pyrithione. Advantageously, the composition consisting of ciclopiroxolamine and piroctone olamine represents the only anti-dandruff active ingredient of the composition.

The invention also relates to a composition comprising as active ingredient a combination comprising ciclopiroxolamine and piroctone olamine, without zinc pyrithione, with at least one dermatologically or cosmetically acceptable excipient, for use thereof in preventing and/or treating seborrhoeic dermatitis.
The invention thus relates to a composition comprising as active ingredient a combination comprising ciclopiroxolamine and piroctone olamine, without zinc pyrithione, with at least one dermatologically or cosmetically acceptable excipient, for use thereof in combating dandruff and/or preventing its formation.

The combination of these two active ingredients has a very interesting anti-dandruff activity, as demonstrated in the examples of the present application, and this through its level of efficacy but also through its rapidity of action. Such a rapidity in the inhibitory effect on M. furfur strains presents an undoubtedly advantageous, and surprising, effect thus enabling a reduced and reasonable exposure time in the context of a topical anti-dandruff treatment.

The combination according to the invention also has a very favourable action on seborrhoeic dermatitis, as demonstrated by its action on M. globosa involved in seborrhoeic dermatitis.

The present invention thus relates to a hair treatment agent or agent consisting of a ciclopiroxolamine and piroctone olamine combination, for the use thereof in treating or preventing dandruff states. The hair treatment agent according to the invention is devoid of zinc pyrithione. In the present description, the ciclopiroxolamine and piroctone olamine combination is likened to a hair treatment agent.

The present invention thus relates to a hair treatment agent or agent consisting of a ciclopiroxolamine and piroctone olamine combination, for the use thereof in treating or preventing seborrhoeic dermatitis. The hair treatment agent according to the invention is devoid of zinc pyrithione. In the present description, the ciclopiroxolamine and piroctone olamine combination is likened to a hair treatment agent.

The invention also relates to a hair treatment agent or agent consisting of a ciclopiroxolamine and piroctone olamine combination, for treating or preventing dandruff states. The hair treatment agent according to the invention is devoid of zinc pyrithione.

The invention also relates to a method for preparing an anti-dandruff composition for topical application on the scalp and/or the hair, the composition comprising a ciclopiroxolamine and piroctone olamine combination as an active anti-dandruff agent, the method comprising the steps of: a) mixing a ciclopiroxolamine and piroctone olamine combination with a dermatologically or cosmetically acceptable vehicle; and b) packaging the mixture resulting from step (a) in an appropriate packaging. Advantageously, the method for producing the composition does not provide for any addition of zinc pyrithione.

Another object of the present invention is the, preferably topical, use of a dermatological or cosmetic (e.g. dermocosmetic) composition comprising the combination of ciclopiroxolamine and piroctone, as defined above, with a dermatologically or cosmetically acceptable excipient, more particularly acceptable for a topical application, in the prevention and/or treatment of dandruff, in particular mild and discreet dandruff.

Another object of the present invention is the, preferably topical, use of a dermatological composition comprising the combination of ciclopiroxolamine and piroctone, as defined above, with a dermatologically acceptable excipient, more particularly acceptable for a topical application, in the prevention and/or treatment of seborrhoeic dermatitis.

Another object of the present invention is a method for preventing and/or treating dandruff comprising the administration, in particular by topical application, of an effective quantity of a dermatological or cosmetic (e.g. dermocosmetic) composition comprising the combination of ciclopiroxolamine and piroctone, as defined above, with a dermatologically or cosmetically acceptable excipient, more particularly acceptable for a topical application, to a person in need. As indicated, advantageously, the ciclopiroxolamine and piroctone olamine combination is the only anti-dandruff active ingredient used in the method according to the invention.

The invention thus relates to a cosmetic method for treating and/or preventing dandruff, in particular mild and discreet dandruff, comprising the administration, in particular by topical application, to the hair or scalp, of a combination according to the invention or of a composition according to the invention.

The present invention also relates to a cosmetic method for treating and/or preventing dandruff, in particular mild and discreet dandruff, comprising the administration, in particular by topical application, of a dermatological or cosmetic (e.g. dermocosmetic) composition comprising the combination of ciclopiroxolamine and piroctone, as defined above, with a dermatologically or cosmetically acceptable excipient, more particularly acceptable for a topical application. As indicated, advantageously, the ciclopiroxolamine and piroctone olamine combination is the only anti-dandruff active ingredient used in the method according to the invention.

In the present invention, “dermatologically or cosmetically acceptable” shall mean that which is usable in the preparation of a dermatological or cosmetic composition, which is generally safe, non-toxic and neither biologically nor otherwise undesirable and which is acceptable for a therapeutic or cosmetic use, in particular by topical application.

The present invention also relates to a method for obtaining anti-dandruff efficacy on the hair or scalp, comprising the steps of—(i) wetting the hair with water; —(ii) applying an effective quantity of an anti-dandruff composition such as described in one of claims 3 to 7 on the hair; —(iii) rinsing the anti-dandruff composition from the hair using water; —(iv) optionally repeating steps (ii) and (iii).

The present invention also relates to a method for killing or slowing the growth of Malassezia spp., in particular M. globosa and/or M. restricta, the method comprising the step of placing Malassezia spp., in particular M. globosa and/or M. restricta in contact with a composition according to the invention that is effective for killing or slowing the growth of Malassezia spp., in particular M. globosa and/or M. restricta.

According to an embodiment, the invention also relates to a method for obtaining anti-seborrhoeic efficacy on the skin, comprising the steps of—(i) optionally wetting the skin with water; —(ii) applying an effective quantity of an anti-seborrhoeic composition such as described in one of claims 3 to 7 on the skin; —(iii) rinsing the anti-seborrhoeic composition from the skin using water; —(iv) optionally repeating steps (ii) and (iii).

In a particular embodiment, the dermatological or cosmetic compositions according to the invention comprise at least one other anti-dandruff active ingredient, different from zinc pyrithione, and in particular an antifungal agent, excluding zinc pyrithione. Advantageously, the at least one other anti-dandruff active ingredient, other than zinc pyrithione, is chosen in the group consisting of pseurotin A, ketoconazole, miconazole, salicylic acid, selenium sulfide, coal tar, azelaic acid, climbazole, undecylenic acid and the mixtures thereof.

The invention preferably relates to compositions, in particular dermatological or cosmetic compositions, in a clean form and suitable for topical application, in particular to the hair and/or the scalp.

The dermatological or cosmetic compositions according to the invention could be present in the usually known forms for topical administration, in other words in particular lotions, shampoos, dry shampoos, conditioners, balms, foam, gels, dispersions, emulsions, sprays, serums, masks, creams and patches.

Advantageously, the compositions according to the invention can be in the usually known forms for a topical administration on the hair and the scalp, in other words in particular a shampoo, dry shampoo, conditioner, hair cream, hair lotion, mask or spray, in particular a leave-in spray.

It is thus distinguished from formulated products that can be rinsed off and formulated products which do not require rinsing.

These compositions generally also contain the combination of ciclopiroxolamine and piroctone olamine according to the present invention, a physiologically acceptable medium, in general based on water or solvent, for example alcohols, ethers or glycols. They can also contain surfactants, complexing agents, preservatives, stabilising agents, emulsifiers, thickeners, gelling agents, humectants, emollients, trace elements, essential oils, perfumes, dyes, moisturizing agents or thermal waters, etc.

Advantageously, the compositions according to the invention comprise 0.01 to 2% by weight, preferably 0.1 to 2% by weight, yet more preferably 0.5 to 2% by weight ciclopiroxolamine, with respect to the total weight of the composition. The composition preferably comprises 1.5% by weight ciclopiroxolamine with respect to the total weight of the composition.

Advantageously, the compositions according to the invention comprise 0.01 to 1% by weight, preferably 0.05 to 1% by weight, yet more preferably 0.1 to 1% by weight piroctone olamine, with respect to the total weight of the composition. The composition preferably comprises 0.3% by weight piroctone olamine with respect to the total weight of the composition.

The compositions of the invention thus advantageously comprise, in % by weight of the final composition, 0.05 to 1% piroctone olamine and 0.1 to 2% ciclopiroxolamine. In particular, a composition according to the invention comprises, in % by weight of the composition, approximately 0.3% piroctone olamine and approximately 1.5% ciclopiroxolamine. More particularly, a composition according to the invention comprises, in % by weight of the composition, approximately 0.15% piroctone olamine and approximately 0.75% ciclopiroxolamine.

The compositions according to the invention comprise ciclopiroxolamine and piroctone olamine in a mass ratio of ciclopiroxolamine:piroctone olamine between 1:10 and 10:1, more particularly between 1:5 and 5:1.

In particular, when expressing the quantities of ciclopiroxolamine and piroctone olamine in the combination according to the invention in parts by weight, this combination comprises between 0.5 and 2 parts piroctone olamine and advantageously between 1 and 10 parts ciclopiroxolamine. The combination according to the invention preferably comprises, in parts by weight, 1 part of piroctone olamine and 5 parts of ciclopiroxolamine.

Such compositions can be produced according to methods well known to a person skilled in the art.

The following examples illustrate the invention, without limiting the scope.

### EXAMPLES

### Example 1: Effects of the Combination of Ciclopiroxolamine and Piroctone Olamine on Inhibiting the Growth of Colonies of Malassezia globosa and restricta

The strains originate from the collection of the Institut Pasteur (Fungi collection, Paris France) for M. globosa (IP2387.96) and from the collection of CBS-KNAW for M. restricta (CBS7877). These strains represent and correspond to the majority of strains involved in dandruff. The strains are incubated for 5 days at 30° C. on modified Dixon agar under aerobic conditions. The inoculation suspensions are prepared in sterile distilled water and are concentrated to 10⁷ CFU/ml. The determination of the minimum inhibitory concentration (MIC) is performed by micromethod in a liquid medium. 100 μl of liquid culture medium is deposited in each well of a sterile 96-well microplate. 100 μl of the solutions to be tested is deposited in the first well of a line of the plate. Dilutions by a factor of 2 are then carried out for wells 1 to 10. The test suspensions of each microorganism are prepared immediately before use in Tryptone salt. 100 μl is deposited in each well of a second microplate, with the exception of column 11. All of the first plate is inoculated using a Denley multipoint inoculate, from the second microplate. After incubation, the MIC are defined as the largest dilution with the absence of visible growth. Columns 11 and 12 serve respectively as a negative and positive control for growth.

The products tested are:
ciclopiroxolamine, at 3% (wt/wt) with sterile distilled water,
piroctone olamine at 0.3% (wt/wt) with 10% ethanol,
ketoconazole (positive control) at 1000 μg/ml (wt/wt) with 10% DMSO,
piroctone olamine/ciclopiroxolamine combinations, with respective concentrations of 0.15% and 0.75%.

The solutions are prepared at the time of the test and used immediately. An ethanol control was produced, solution at 10%.

The determination of the minimum fungicide concentrations (MFC) is carried out by subculturing the MIC microplates on agar medium, using the Denley multipoint inoculator. After incubation, the MFC are defined as the largest dilution with the absence of visible growth. All the tests were carried out in duplicate.

The synergistic effect of two products is determined by calculating the synergy index (FIC, Fractional Inhibitory Concentration).

FIC Index=(MIC product A combined)/(MIC product alone)+(MIC product B combined)/(MIC product B alone).

A synergy is revealed when the value of the FIC index is <0.5.

### Results

Tables 1 (Malassezia globosa) and 2 (Malassezia restricta) below indicate the values of MIC and MFC in percentage (wt/wt) and in mg/1 for the products alone and the combination.

The maximum test concentration corresponds to the concentration of the parent solution/2.

TABLE 1

| | M. globosa | | | |
|---|---|---|---|---|
| | MIC | | MFC | |
| | % | mg/l | % | mg/l |
| PO | 2.3 × 10⁻³ | 23 | 2.3 × 10⁻³ | 23 |
| CPO | 2.9 × 10⁻³ | 29 | 2.9 × 10⁻³ | 29 |
| PO/CPO | 7.32 × 10⁻⁵/ | 0.732/3.65 | 7.32 × 10⁻⁵/ | 0.732/3.65 |
| | 3.65 × 10⁻⁴ | | 3.65 × 10⁻⁴ | |
| Keto | | 0.98 | | 1.94 |

PO: piroctone olamine;
CPO: ciclopiroxolamine;
Keto: ketoconazole

TABLE 2

| | M. restricta | | | |
|---|---|---|---|---|
| | MIC | | MFC | |
| | % | mg/l | % | mg/l |
| PO | 2.3 × 10⁻³ | 23 | 2.3 × 10⁻³ | 23 |
| CPO | 1.5 × 10⁻³ | 15 | 1.5 × 10⁻³ | 15 |
| PO/CPO | 7.32 × 10⁻⁵/ | 0.732/3.65 | 7.32 × 10⁻⁵/ | 0.732/3.65 |
| | 3.65 × 10⁻⁴ | | 3.65 × 10⁻⁴ | |
| Keto | | 0.49 | | 0.49-0.98. |

PO: piroctone olamine;
CPO: ciclopiroxolamine;
Keto: ketoconazole

No activity was noted for ethanol under the test conditions.

Tables 1 and 2 show that piroctone olamine and ciclopiroxolamine alone exert a strong inhibitory activity, as does ketoconazole which serves as a positive control. The most significant result is found with the combination of the two products; indeed a synergistic inhibitory effect is shown with a reduction in the MIC by a factor of 10 to 100 compared with the MIC of the products alone. Results for the two strains tested are entirely comparable.

A synergy between piroctone olamine and ciclopiroxolamine is indeed very clearly demonstrated when the synergy index is calculated, which reaches only 0.16 for Malassezia globosa and 0.28 for Malassezia restricta.

This synergy between piroctone olamine and ciclopiroxolamine, in particular for M. globosa, also enables a beneficial effect to be demonstrated for seborrhoeic dermatitis.

### Example 2: Effects of the Combination of Ciclopiroxolamine and Piroctone Olamine on Anti-Malassezia Activity

The capacity of piroctone olamine and of ciclopiroxolamine to inhibit the growth of colonies of Malassezia is significant but insufficient to predict a lethal activity for Malassezia and consequently an optimum elimination of dandruff. Indeed, Malassezia can proliferate again if it is not entirely or sufficiently eliminated. Consequently, the effectiveness of a dandruff active ingredient must also be shown by demonstrating its effect on the lethality for Malassezia. This component is generally measured by estimating the decrease in microbial colonies over time.

In the context of this work, the model has been optimised in terms of sample preparation, contact time and sample evaluation concentration.

The aim of the test described below is to evaluate, in a relevant manner, the anti Malassezia (M. globosa and M. restricta) activity of piroctone olamine, ciclopiroxolamine and their combination and to compare it with that of ketoconazole.

The products to be tested alone and combined at the defined concentrations are placed in contact with a suspension of Malassezia containing 10⁷ CFU/ml for a contact time of 5 minutes, 15 minutes and 30 minutes. After each contact time, a sample is taken, then diluted and filtered on a cellulose membrane. Each membrane is then deposited on the agar medium (Dixon) and incubated at 30° C. After incubation, the colonies are counted.

The products tested are:
ciclopiroxolamine, at 1.5% (wt/wt) diluted with sterile distilled water,
piroctone olamine at 0.3% (wt/wt) dissolved in ethanol, then addition of water for injectable preparations (final ethanol concentration of 9%),
ketoconazole (positive control) at 2% (wt/wt) dissolved in DMSO, then addition of water for injectable preparations (final concentration of DMSO 9%).
piroctone olamine/ciclopiroxolamine combinations, with respective concentrations of 0.3% and 1.5%.

The solutions are prepared at the time of the test and used immediately.

### Results

The results obtained with these different products are presented in tables 3 (Malassezia globosa) and 4 (Malassezia restricta) below.

TABLE 3
Fungicidal activity of active ingredients on Malassezia globosa

| | Log Reduction with different contact times | | |
|---|---|---|---|
| | 5 minutes | 15 minutes | 30 minutes |
| piroctone olamine (PO) | 0.36 | 0.61 | 1.24 |
| ciclopiroxolamine (CPO) | 2.45 | 3.88 | >4.22 |
| PO/CPO | >4.27 | >4.27 | >4.27 |
| ketoconazole | 1.47 | 1.29 | 1.19 |

TABLE 4
Fungicidal activity of the active ingredients on Malassezia restricta

| | Log Reduction with different contact times | | |
|---|---|---|---|
| | 5 minutes | 15 minutes | 30 minutes |
| piroctone olamine (PO) | 1.19 | 1.28 | 1.73 |
| ciclopiroxolamine (CPO) | 1.99 | 3.3 | >4.17 |
| PO/CPO | >4.44 | >4.44 | >4.44 |
| ketoconazole | 0.85 | 1.12 | 1.09 |

Ketoconazole has an activity on Malassezia globosa and restricta at the three contact times investigated, this activity, although modest, enables this test to be confirmed.

Piroctone olamine and ciclopiroxolamine show an anti Malassezia activity greater than that found with ketoconazole. Surprisingly, the combination of piroctone olamine with ciclopiroxolamine proves even more effective, in particular at the contact time of 5 minutes. Indeed, this combination makes it possible to obtain a percentage reduction in the number of viable cells of Malassezia globosa or Malassezia restricta greater than 99.99% after 5 minutes of contact. The inventors thus demonstrated a synergy between piroctone olamine and ciclopiroxolamine on the two strains tested. The combination of these two active ingredients thus exhibits a very interesting anti-dandruff activity, through its level of effectiveness but also through its rapidity of action which exhibits a net advantageous effect enabling a reduced and reasonable exposure time in the context of anti-dandruff treatment.

1-13. (canceled)

14. Method for preventing and/or treating seborrhoeic dermatitis and/or dandruff comprising the administration of an effective amount of a combination comprising ciclopiroxolamine and piroctone olamine, without zinc pyrithione, to a patient in need thereof.

15. Method according to claim 14 wherein the combination is administered via the topical route.

16. Method according to claim 14 wherein the combination is administered by topical application to the hair or on the scalp.

17. Method for preventing and/or treating seborrhoeic dermatitis and/or dandruff comprising the administration of an effective amount of a composition comprising, as active ingredient, a combination comprising ciclopiroxolamine and piroctone olamine, without zinc pyrithione, with at least one dermatologically or cosmetically acceptable excipient, to a patient in need thereof.

18. Method according to claim 17 wherein the composition comprises 0.01 to 2% by weight ciclopiroxolamine with respect to the total weight of the composition.

19. Method according to claim 17 wherein the composition comprises 0.01 to 1% by weight piroctone olamine with respect to the total weight of the composition.

20. Method according to claim 17 wherein the composition is administered via the topical route.

21. Method according to claim 17 wherein the combination is administered by topical application on the scalp.

22. Method according to claim 17 wherein the composition further comprises another anti-dandruff active ingredient.

23. Method for obtaining an anti-dandruff efficacy on the hair or scalp, comprising the steps of
(i) wetting the hair with water;
(ii) applying an effective quantity of an anti-dandruff composition such as described in claim 17 on the hair;
(iii) rinsing the anti-dandruff composition from the hair with water;
(iv) optionally, repeating steps (ii) and (iii).

24. Method for obtaining an anti-seborrhoeic efficacy on the skin, comprising the steps of
(i) optionally wetting the skin with water;
(ii) applying an effective quantity of an anti-seborrhoeic composition such as described in claim 17 on the skin;
(iii) rinsing the anti-seborrhoeic composition from the skin with water;
(iv) optionally, repeating steps (ii) and (iii).
